(19) United States
(12) Patent Application Publication
(10) Pub. No.: US 2010/0316586 A1
(43) Pub. Date: Dec. 16, 2010

(54) STYLING AGENT FOR KERATIN FIBERS

(75) Inventors: Thorsten Knappe, Schenefeld (DE); Ina Krügermann, Langenfeld (DE); Johann Klein, Dusseldorf (DE)

Correspondence Address: INGRASSIA FISHER & LORENZ, P.C., 7010 E. COCHISE ROAD, SCOTTSDALE, AZ, 85253, US

(73) Assignee: HENKEL AG & CO. KGAA, Dusseldorf (DE)

(21) Appl. No.: 12/514,419

(22) PCT Filed: Sep. 4, 2007
(86) PCT No.: PCT/EP07/59205

§ 371 Date: May 11, 2009

(30) Foreign Application Priority Data
Dec. 8, 2006 (DE) 10-2006-058390.6

(51) Int. Cl. A61K 8/81; A61Q 5/06

(52) U.S. Cl. 424/70.16

(57) ABSTRACT

The invention relates to an agent for shaping keratin fibers, containing at least one bonding agent selected from acrylic acid ester copolymers and methacrylic acid ester copolymers in a cosmetically acceptable carrier and to the use of corresponding bonding agents in styling agents for introducing or improving a remodeling capability of a hairstyle that has been created or fixed using said styling agent.

### CROSS REFERENCED TO RELATED APPLICATION

This application is a U.S. National Stage entry under 35 U.S.C §371 based on International Application No. PCT/EP2007/059205, filed 4 Sep. 2007, which was published under PCT Article 21(2) and claims the benefit of the filing date of German Patent Application No 102006058390.6 filed 8 Dec. 2006, the disclosures of which applications are hereby incorporated herein by reference.

### TECHNICAL FIELD

The present invention relates generally to cosmetic compositions and the use of cosmetic compositions, and more particularly relates to agents for shaping keratin fibers, containing in a cosmetically acceptable carrier at least one specific pressure-sensitive adhesive, and to the use of corresponding pressure-sensitive adhesives in styling agents for bringing about or improving remodelability of a hairstyle created or set with the styling agent.

### BACKGROUND

Keratin fibers are in principle taken to mean all kinds of animal hair, for example wool, horsehair, angora hair, furs, feathers and products or textiles manufactured therefrom. Preferably, however, the keratin fibers comprise human hair.

An attractive hairstyle is today generally regarded as an indispensable part of a well-groomed appearance. Current fashion trends often mean that, with many hair types, hairstyles which are considered smart can only be achieved or maintained for an extended period by using setting active ingredients or targeted modification of hair structure. Hair treatment agents which bring about permanent or temporary shaping of the hair accordingly play an important role.

Keratin-containing fibers are conventionally permanently shaped by mechanically shaping the fibers and optionally setting the shape by suitable auxiliaries. The fibers are treated with a keratin-reducing preparation before and/or after this shaping. Some of the disulfide bridges of the keratin molecule are here cleaved, so making the keratin fibers more flexible. After rinsing, the fibers are then treated in the “setting step” with an oxidizing agent preparation, disulfide bridges in the hair keratin being relinked, such that the keratin structure is fixed in the given shape. Permanent shaping is obtained which readily withstands conventional environmental influences, such as hair washing for example. A disadvantage of this procedure, however, is that the natural structure of the treated fibers is inevitably modified. This often results in weakening of the fibers. In particular when permanent shaping is repeated or further treatments which stress the fibers, such as for example oxidative dyeing or bleaching, are carried out, it is not possible to rule out damage to the fibers. Moreover, once set, the shape cannot be modified without another permanent shaping or at least applying a temporary styling agent. However, there is precisely a desire among consumers for hairstyling which does not damage the hair and additionally offers a degree of flexibility.

One gentle method of shaping keratin-containing fibers is temporary shaping, as is achieved for example by conventional hairsprays, hair waxes, hair gels, setting lotions etc.

Appropriate agents for temporary shaping conventionally contain synthetic polymers as the shaping component. Preparations which contain a dissolved or dispersed polymer may be applied to the hair by means of propellant gases or by a pump mechanism. Hair gels and hair waxes in particular are, however, not generally applied directly onto the hair, but are rather distributed in the hair by means of a comb or the hands.

One essential characteristic of an agent for shaping keratin fibers, hereinafter also designated “styling agent”, is to provide the strongest possible hold for treated fibers in the shape created. If the keratin fibers are human hair, this is thus referred to as strong styling hold. Furthermore, there is an increasing expectation among users of a styling agent that the created hairstyle should remain shapeable without the hold being lost due to reshaping. This characteristic is also known as remodelability of a hairstyle. Moisture resistance often also plays a part here. Once created, the hairstyle should also be resistant to high levels of atmospheric humidity and to perspiration.

Available styling agents which impart good hold to the hairstyle are generally based on film-forming and/or setting polymers. Strength of hold may be adjusted by suitable selection of the type and/or concentration of the film-forming and/or setting polymer. The polymers form a film on the treated fibers and crosslink individual fibers to one another, such that the fibers are fixed in a given position. If, once a hairstyle has been fixed in place, it is subjected to mechanical loading, the polymer films are easily broken, crosslinks are irreversibly released and fiber hold is lost. Since reshaping of the hairstyle is inevitably accompanied by mechanical loading, such styling agents do not permit remodelability of the hairstyle.

Available styling agents which permit a certain remodelability, for instance hair waxes or corresponding pastes, are generally based on waxes and have the disadvantage that the achievable degree of hold is slight.

Moreover, it is not possible to achieve permanent effects with known temporary shaping agents. The agents are washed out when the fibers are subjected to normal washing. Renewed shaping in turn entails the use of corresponding styling agents.

Accordingly, it is desirable to provide an agent for shaping keratin fibers which is distinguished by good setting performance, i.e. it imparts a strong hold to the treated fibers, permits remodelability of the fibers without the hold being lost as a consequence and furthermore permits gentle permanent shaping.

### DESCRIPTION OF THE DRAWINGS

The present invention will hereinafter be described in conjunction with the following drawing figures, wherein like numerals denote like elements, and wherein:

FIG. 1 is an illustration of the structural formula of cationic polymer that can be used in an embodiment of the present invention;

FIG. 2 is an illustration of the structural formula of monomers having quaternary ammonium groups that can be used to form amphoteric polymers useful in an embodiment of the present invention;

FIG. 3 is an illustration of the structural formula of monomeric carboxylic acids that can be used to form amphoteric polymers useful in an embodiment of the present invention;

FIG. 4 is an illustration of the structural formula of dicarboxylic acids useful in an embodiment of the present invention;

FIG. 5 is an illustration of the structural formula of an ectoin or ectoin derivative useful in an embodiment of the present invention;

FIG. 6 is an illustration of the structural formula of another ectoin or ectoin derivative useful in an embodiment of the present invention;

FIG. 7 is an illustration of the structural formula of fatty acid partial glycerides useful in an embodiment of the present invention; and

FIGS. 8-16 are illustrations of the structural formulas of cationic direct-absorbing dyes useful in an embodiment of the present invention.

### BRIEF SUMMARY

An agent for shaping keratin fibers and a method for shaping keratin fibers is provided. In accordance with an exemplary embodiment of the present invention, the agent contains in a cosmetically acceptable carrier at least one pressure-sensitive adhesive selected from acrylic acid ester copolymers and methacrylic acid ester copolymers is provided.

A method for shaping keratin fibers is provided in accordance with another exemplary embodiment. The method comprises the steps of applying to the keratin fibers an agent comprising a pressure sensitive adhesive selected from acrylic acid ester copolymers and methacrylic acid ester copolymers.

### DETAILED DESCRIPTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Surprisingly, an agent for shaping keratin fibers which is distinguished by good setting performance, permits remodelability of the fibers without the hold being lost as a consequence and furthermore permits greater permanent shaping may be achieved by using specific pressure-sensitive adhesives in styling agents.

Accordingly, in one exemplary embodiment, an agent for shaping keratin fibers contains in a cosmetically acceptable carrier at least one pressure-sensitive adhesive selected from acrylic acid ester copolymers and methacrylic acid ester copolymers.

Pressure-sensitive adhesives are taken to mean adhesives which are sensitive to pressure, are permanently tacky and retain adhesive properties in solvent-free form at 20° C. and, with little substrate specificity, adhere immediately to almost all substrates when exposed to slight pressure. Suitable pressure-sensitive adhesives are those which form adhesive bonds which can be released without destroying the adhesively bonded substrates. Many such pressure-sensitive adhesives are known and commercially available from the adhesives industry, where they are conventionally used for producing adhesive tapes, adhesive films, self-adhesive labels, self-adhesive notes (i.e., Post-it® notes) and similar products.

In accordance with an exemplary embodiment, the acrylic acid ester copolymers and methacrylic acid ester copolymers used preferably comprise copolymers of the esters of the particular acids with non-tertiary alkyl alcohols having alkyl residues with 1 to 12 carbon atoms, in particular 2 to 4 carbon atoms. Suitable monomers which may be mentioned by way of example are ethyl acrylate, ethyl methacrylate, propyl acrylate, propyl methacrylate, n-butyl acrylate, n-butyl methacrylate, isobutyl acrylate, 2-methylbutyl acrylate, 2-ethylhexyl acrylate, n-octyl acrylate, isooctyl acrylate, isooctyl methacrylate, isononyl acrylate and isodecyl acrylate.

The pressure-sensitive adhesives contemplated herein are those which are not removed from the treated keratin fibers virtually or completely without leaving any residue after just one treatment with water or a conventional commercial shampoo. Removal of the pressure-sensitive adhesives virtually or completely without leaving any residue should here be taken to mean that the washed and then dried fibers neither feel tacky, nor, in comparison with a reference fiber which has not been treated with the agent, exhibit improved stability of mechanical shaping. Despite washing, the pressure-sensitive adhesive used thus remains in sufficient quantity on the keratin fibers to impart hold to the fibers even after washing and to permit renewed shaping of the fibers without any further auxiliaries having to be applied onto the fibers. In a preferred embodiment, the pressure-sensitive adhesives are those which also withstand repeated washing of the fibers, preferably at least 2 washings, particularly preferably at least 4 washings. In this manner, agents are obtained which permit long-lasting or, in extreme cases, even permanent shaping or shapeability of the treated fibers.

In a preferred embodiment, the agents accordingly contain pressure-sensitive adhesives which remain on the fibers even after rinsing the fibers twice with water at a temperature of 30° C. in each case for 2 minutes, the water being used in each rinsing operation in a quantity such that the weight ratio of fibers to water amounts to 1:10.

The agents preferably contain a pressure-sensitive adhesive selected from butyl acrylate/butadiene copolymers.

The butyl acrylate/butadiene copolymer used is preferably the copolymer of butyl acrylate and butadiene distributed by Dow Chemical Company of Midland, Mich. under the name Ucar Latex XZ 91964. This comprises a dispersion of the copolymer blended with natural rubber.

Further preferred acrylic acid ester copolymers are the commercially obtainable products from Avery Dennison of Pasadena, Calif., which are offered for sale under the names Polytex 6385 and Polytex 6351.

In an exemplary embodiment, the total quantity of pressure-sensitive adhesives in the agents amounts to 0.1 to 10 weight percent (wt. %), preferably 0.5 to 8 wt. %. The percentages relate to the total agent, less any propellants optionally present in the agent.

It goes without saying that the agents may also contain a mixture of a plurality of the stated pressure-sensitive adhesives, the total quantity preferably not exceeding 10 wt. %.

In an exemplary embodiment, the pressure-sensitive adhesives are preferably introduced into the agents in the form of a solution or a dispersion, the solvent or dispersing medium used preferably being water, alcohol, in particular ethanol and isopropanol, and mixtures thereof.

It is also possible to add further pressure-sensitive adhesives, it being, however, preferred for the agents to contain no further pressure-sensitive adhesives in addition to the pressure-sensitive adhesives selected from acrylic acid ester copolymers and methacrylic acid estercopolymers.

In another exemplary embodiment, the agents may furthermore contain at least one film-forming and/or setting polymer other than the pressure-sensitive adhesive. It is, however, also possible to dispense with the addition of such further film-forming and/or setting polymers.

The total quantity of film-forming and/or setting polymers should amount to 0.1 to 20 wt. %, preferably 0.5 to 15 wt. %, more preferably from 1.0 to 10 wt. %.

These film-forming and/or setting polymers may be both permanently and temporarily cationic, anionic, nonionic or amphoteric. When using at least two film-forming and/or setting polymers, it goes without saying that these may have different charges. It may be preferable for an ionic film-forming and/or setting polymer to be used together with an amphoteric and/or nonionic film-forming and/or setting polymer. The use of at least two oppositely charged film-forming and/or setting polymers is also preferred. In the latter case, a particular embodiment may in turn additionally contain at least one further amphoteric and/or nonionic film-forming and/or setting polymer.

Since polymers are often multifunctional, their functions cannot always be clearly and unambiguously delimited from one another. This is true in particular of film-forming and setting polymers. Some film-forming polymers will nevertheless be described by way of example. It should however be explicitly pointed out at this point that both film-forming and setting polymers are essential for the purposes of the present invention. Since the two properties are also not wholly mutually independent, the term “setting polymers” should always be understood also to mean “film-forming polymers” and vice versa.

The preferred properties of film-forming polymers include film formation. Film-forming polymers should be understood to mean those polymers which, on drying, leave behind a continuous film on the skin, hair or nails. Such film formers may be used in the most varied of cosmetic products, such as for example face masks, make-up, hair setting preparations, hairsprays, hair gels, hair waxes, hair tonics, shampoos or nail polishes. Preferred polymers are in particular those which exhibit sufficient solubility in alcohol or water/alcohol mixtures to be present in the agent in completely dissolved form. The film-forming polymers may be of synthetic or natural origin.

Film-forming polymers are further understood according to the invention to mean those polymers which are capable, when applied in a 0.01 to 20 wt. % aqueous, alcoholic or aqueous/alcoholic solution, of depositing a transparent polymer film on the hair. The film-forming polymers may be charged in any one of an anionic, amphoteric, nonionic, permanently cationic or temporarily cationic manner.

Suitable synthetic, film-forming, hair setting polymers are homo- or copolymers synthesized from at least one of the following monomers: vinylpyrrolidone, vinylcaprolactam, vinyl esters such as for example vinyl acetate, vinyl alcohol, acrylamide, methacrylamide, alkyl- and dialkylacrylamide, alkyl- and dialkylmethacrylamide, alkyl acrylate, alkyl methacrylate, propylene glycol or ethylene glycol, the alkyl groups of these monomers preferably being C₁ to C₇ alkyl groups, particularly preferably C₁ to C₃ alkyl groups.

Examples which may be mentioned are homopolymers of vinylcaprolactam, vinylpyrrolidone or N-vinylformamide. Examples of further suitable synthetic film-forming, hair setting polymers are copolymers of vinylpyrrolidone and vinyl acetate, terpolymers of vinylpyrrolidone, vinyl acetate and vinyl propionate, polyacrylamides, which are distributed for example by CHEM-Y GmbH of Germany, under the tradename Akypomine® P 191, or by Seppic of Fanfield, N.J., under the tradename Sepigel 305®; polyvinyl alcohols, which are sold for example by DuPont of Wilmington, Del., under the tradename Elvanol® or by Air Products and Chemicals Inc. of Allentown, Pa., under the tradename Vinol® 523/540 as well as polyethylene glycol/polypropylene glycol copolymers, which are sold, for example, by Union Carbide Corporation of Houston, Tex., under the tradename Ucon®.

Examples of suitable natural film-forming polymers are cellulose derivatives, for example hydroxypropylcellulose with a molecular weight of 30,000 to 50,000 g/mol, which is distributed for example by Lehmann & Voss, Germany under the tradename Nisso SI®.

Setting polymers assist in holding or building up the volume and fullness of the overall hairstyle. These “setting” polymers are simultaneously also film-forming polymers and therefore are generally typical substances for shaping hair treatment agents such as hair setting preparations, hair mousses, hair waxes, hairsprays. Film formation may in this respect take place only at points and connect only a few fibers together.

Substances which additionally lend the hair hydrophobic properties are preferred here, because they reduce the tendency of the hair to absorb moisture, i.e. water. This reduces the tendency of hair strands to hang down limply and thus ensures that a hairstyle retains its structure for a long time. The “curl retention” test is often used as a test method in such cases. These polymeric substances may additionally be successfully incorporated into leave-on and rinse-off hair tonics or shampoos. Since polymers are often multifunctional, i.e. have a plurality of applicationally desirable effects, many polymers are classified in a plurality of groups according to their mode of action, for example also in the CTFA Handbook. Because of the significance specifically of the setting polymers, these will be listed below explicitly by their INCI names. This list of polymers which are preferably to be used obviously thus also includes precisely the stated film-forming polymers.

Examples of conventional film-forming, setting polymers are Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/VP Copolymer, Adipic Acid/Diethylene-triamine Copolymer, Adipic Acid/Dimethylaminohydroxypropyl Diethylene-triamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Amino-ethylpropanediol-AMPD-Acrylates/Diacetoneacrylamide Copolymer, Ammonium VA/Acrylates Copolymer, AM PD-Acrylates/Diacetoneacrylamide Copolymer, AMP-Acrylates/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethyl-methacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-Hydroxypropyl Dimethicone Acrylate Copolymer, Isopropyl Ester of PVM/MA Copolymer, Lauryl Acrylate Crosspolymer, Lauryl Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVP/VA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylamino-ethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VPA/A Copolymer, VP/Vinyl Caprolactam/DMAPA Acrylates Copolymer, and Yeast Palmitate.

In a preferred embodiment, the agent contains at least one film-forming and/or setting polymer selected from:
aminomethylpropanol salts of copolymers of allyl methacrylate with one or more monomers selected from acrylic acid, methacrylic acid, acrylic acid esters and methacrylic acid esters;
vinylpyrrolidone/vinyl acetate copolymers;
vinylpyrrolidone/vinylcaprolactam/dimethylaminopropylacrylamide copolymers;
copolymers of octylacrylamide with t-butylaminoethyl methacrylate and two or more monomers selected from acrylic acid, methacrylic acid, acrylic acid esters and methacrylic acid esters; and
copolymers of C₁₋₂ alkyl succinates with hydroxyalkyl acrylates and one or more monomers selected from acrylic acid, methacrylic acid, acrylic acid esters and methacrylic acid esters.

Corresponding film-forming and/or setting polymers are commercially obtainable.

The stated acrylic acid esters and methacrylic acid esters preferably comprise C₁-C₁₂ alkyl acrylates and C₁-C₁₂ alkyl methacrylates, more preferably methyl acrylate, ethyl acrylate, propyl acrylate, methyl methacrylate, ethyl methacrylate, propyl methacrylate and mixtures thereof.

The preferably used aminomethylpropanol salt of copolymers of allyl methacrylate with one or more monomers selected from acrylic acid, methacrylic acid, acrylic acid esters and methacrylic acid esters is the copolymer with the INCI name AMP-Acrylates/Allyl Methacrylate Copolymer which is distributed by Noveon of Wickliffe, Ohio, under the name Fixate™ G-100. In a preferred embodiment, the agent contains this copolymer.

A preferred vinylpyrrolidone/vinyl acetate copolymer is the PVP/VA copolymer 60-40 W (INCI name: VPA/A Copolymer, Aqua, Laurtrimonium Chloride).

A vinylpyrrolidone/vinylcaprolactam/dimethylaminopropylacrylamide copolymer which is preferably used is the copolymer with the INCI name VP/Vinyl Caprolactam/DMAPA Acrylates Copolymer obtainable from International Specialty Products (ISP) of Columbia, Md., under the name Aquaflex SF 40.

A preferred copolymer of octylacrylamide with t-butylaminoethyl methacrylate and two or more monomers selected from acrylic acid, methacrylic acid, acrylic acid esters and methacrylic acid esters is the copolymer with the INCI name Octylacrylamide/Acrylates Butylaminoethyl Methacrylates Copolymer obtainable from National Starch Company of Bridgewater, N.J. under the name Amphomer®.

A preferred copolymer of C₁₋₂ alkyl succinate with hydroxyalkyl acrylates and one or more monomers selected from acrylic acid, methacrylic acid, acrylic acid esters and methacrylic acid esters is the copolymer with the INCI name Acrylates/C1-2 Succinates/Hydroxyacrylates Copolymer obtainable from ISP under the name Allianz™ LT 120.

In an exemplary embodiment, the agents may furthermore contain conditioning substances. Protein hydrolysates and/or the derivatives thereof are, for example, suitable.

Protein hydrolysates are product mixtures which are obtained by acidically, basically or enzymatically catalysed degradation of proteins. As used herein, the term protein hydrolysates also covers total hydrolysates and individual amino acids and the derivatives thereof and mixtures of different amino acids. Furthermore, polymers built up from amino acids and amino acid derivatives are also covered herein by the term protein hydrolysates. The latter include for example polyalanine, polyasparagine, polyserine etc. Further examples of compounds which may be used herein are L-alanyl-L-proline, polyglycine, glycyl-L-glutamine or D/L-methionine-5-methylsulfonium chloride. It goes without saying that β-amino acids and the derivatives thereof such as β-alanine, anthranilic acid or hippuric acid may also be used. In one exemplary embodiment, the molecular weight of the protein hydrolysates which may be used is between about 75, the molecular weight of glycine, and about 200,000 daltons, preferably about 75 to about 50,000 daltons and more preferably to about 75 to about 20,000 daltons.

The agents may use protein hydrolysates of plant, animal, marine. or synthetic origin.

Animal protein hydrolysates are for example elastin, collagen, keratin, silk and milk protein hydrolysates which may also assume salt form. Such products are distributed for example under the tradenames Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

The use of silk protein hydrolysates is of particular interest. Silk is taken to mean the fibers of the cocoon of the silkworm (Bombyx mori L). Raw silk fiber consists of a double filament fibroin. Sericin acts as the cement holding this double filament together. Silk is made up of 70-80 wt. % fibroin, 19-28 wt. % sericin, 0.5-1 wt. % fat and 0.5-1 wt. % colors and mineral constituents.

Hydroxyamino acids, accounting for approximately 46 wt. %, are the substantial constituents of sericin. Sericin consists of a group of 5 to 6 proteins. The substantial amino acids of sericin are serine (Ser, 37 wt. %), aspartate (Asp, 26 wt. %), glycine (Gly, 17 wt. %), alanine (Ala), leucine (Leu) and tyrosine (Tyr).

Fibroin, which is insoluble in water, should be classed among scleroproteins with a long-chain molecular structure. The main constituents of fibroin are glycine (44 wt. %), alanine (26 wt. %) and tyrosine (13 wt. %). Another substantial structural feature of fibroin is the hexapeptide sequence Ser-Gly-Ala-Gly-Ala-Gly.

It is technically straightforward to separate the two silk proteins from one another. It is thus not surprising that both sericin and fibroin are in each case individually known as raw materials for use in cosmetic products. Protein hydrolysates and derivatives based on the individual silk proteins are furthermore known raw materials in cosmetics. For example, sericin as such is distributed by Pentapharm Ltd. of Switzerland, as a commercial product known as Sericin Code 303-02. Fibroin is still more frequently offered for sale as a protein hydrolysate with various molecular weights. These hydrolysates are in particular distributed as “silk hydrolysates”. Hydrolysed fibroin with average molecular weights of between 350 and 1000 is, for example, accordingly distributed under the tradename Promois® Silk. German patent DE 31 39 438 A1 also describes colloidal fibroin solutions as an additive in cosmetics.

The positive properties of the silk protein derivatives of sericin and fibroin are in each case per se known in the literature. The cosmetic effects of sericin on the skin have been described as irritation-relieving, hydrating and film-forming. A fibroin derivative is described, for example in German patent DE 31 39 438 A1, as having a conditioning and softening action on hair. According to German patent DE 102 40 757 A1, a synergistic enhancement of the positive effects of the silk proteins and the derivatives thereof may be achieved by simultaneously using sericin and fibroin or the derivatives and/or hydrolysates thereof.

In one embodiment, the silk protein hydrolysate used in the agent is thus preferably an active ingredient complex (A) consisting of active ingredient (A1) selected from sericin, sericin hydrolysates and/or the derivatives thereof, and mixtures thereof, and an active ingredient (A2) selected from fibroin, and/or fibroin hydrolysates and/or the derivatives thereof and/or mixtures thereof.

The active ingredient complex (A) used brings about a significant, synergistic improvement in the above-described essential internal and external structural features and in the strength and elasticity of human hair.

Active ingredients (A1) which may be used in the active ingredient complex (A) are:
native sericin;
hydrolysed and/or further derivatized sericin, such as for example commercial products with the INCI names Sericin, Hydrolyzed Sericin, or Hydrolyzed Silk;
a mixture of the amino acids serine, aspartate and glycine and/or the methyl, propyl, iso-propyl, butyl, iso-butyl esters thereof, the salts thereof such as for example hydrochlorides, sulfates, acetates, citrates, tartrates, this mixture containing the serine and/or the derivatives thereof in an amount of about 20 to about 60 wt. %, the aspartate and/or the derivatives thereof in an amount of about 10 to about 40 wt. % and the glycine and/or the derivatives thereof in an amount of about 5 to about 30 wt. %, with the proviso that the quantities of these amino acids and/or the derivatives thereof preferably add up to 100 wt. %; and
mixtures thereof.

Active ingredients (A2) which may be used in the active ingredient complex (A) are:
native fibroin converted into a soluble form;
hydrolysed and/or further derivatized fibroin, in particular partially-hydrolysed fibroin, which contains the amino acid sequence Ser-Gly-Ala-Gly-Ala-Gly as the main constituent;
the amino acid sequence Ser-Gly-Ala-Gly-Ala-Gly;
a mixture of the amino acids glycine, alanine and tyrosine and/or the methyl, propyl, iso-propyl, butyl, iso-butyl esters thereof, the salts thereof such as for example hydrochlorides, sulfates, acetates, citrates, tartrates, this mixture containing the glycine and/or the derivatives thereof in an amount of about 20 to about 60 wt. %, the alanine and/or the derivatives thereof in an amount of about 10 to about 40 wt. % and the tyrosine and/or the derivatives thereof in an amount of about 0 to about 25 wt. %, with the proviso that the quantities of these amino acids and/or the derivatives thereof preferably add up to 100 wt. %; and
mixtures thereof.

Particularly good conditioning properties may be achieved if one of the two active ingredient components of the active ingredient complex (A) is used in the native or at most solubilized form. It is also possible to use a mixture of a plurality of active ingredients (A1) and/or (A2).

It may be preferred for the two active ingredients (A1) and (A2) to be used in the ratio of about 10:90 to about 70:30, preferably about 15:85 to about 50:50 and more preferably about 20:80 to about 40:60, relative to the respective contents of active substance in the products.

The derivatives of hydrolysates of sericin and fibroin comprise both anionic and cationized protein hydrolysates. The protein hydrolysates of sericin and fibroin and the derivatives produced therefrom may be obtained from the corresponding proteins by chemical, in particular alkaline or acidic, hydrolysis, by enzymatic hydrolysis and/or a combination of both types of hydrolysis. Protein hydrolysis as a rule gives rise to a protein hydrolysate with a molecular weight distribution of approximately 100 daltons up to several thousand daltons. Preferred protein hydrolysates of sericin and fibroin and/or the derivatives thereof are those, the underlying protein moiety of which has a molecular weight of about 100 up to about 25000 daltons, preferably about 250 to about 10000 daltons. Cationic protein hydrolysates of sericin and fibroin should furthermore also be taken to mean quaternized amino acids and mixtures thereof. Quaternization of the protein hydrolysates or of the amino acids is often performed by means of quaternary ammonium salts such as for example N,N-dimethyl-N-(n-alkyl)-N-(2-hydroxy-3-chloro-n-propyl)-ammonium halides. The cationic protein hydrolysates may additionally be still further derivatized. Typical examples of the cationic protein hydrolysates and derivatives usable in the agent are those that are commercially obtainable and mentioned under the INCI names in the “International Cosmetic Ingredient Dictionary and Handbook”, (seventh edition 1997, The Cosmetic, Toiletry, and Fragrance Association 1101 17th Street, N.W., Suite 300, Washington, D.C. 20036-4702): Cocodimonium Hydroxypropyl Hydrolyzed Silk, Cocodimonium Hydroxypropyl Silk Amino Acids, Hydroxypropyltrimonium Hydrolyzed Silk, Lauryldimonium Hydroxypropyl Hydrolyzed Silk, Steardimonium Hydroxypropyl Hydrolyzed Silk, Quaternium-79 Hydrolyzed Silk. Typical examples of the anionic protein hydrolysates and derivatives which may be mentioned are those that are commercially obtainable and mentioned under the INCI names in the “International Cosmetic Ingredient Dictionary and Handbook”, (seventh edition 1997, The Cosmetic, Toiletry, and Fragrance Association 1101 17th Street, N.W., Suite 300, Washington, D.C. 20036-4702): Potassium Cocoyl Hydrolyzed Silk, Sodium Lauroyl Hydrolyzed Silk or Sodium Stearoyl Hydrolyzed Silk. Finally, typical examples of the derivatives of sericin and fibroin which may be mentioned are the commercially obtainable products known by the INCI names: Ethyl Ester of Hydrolyzed Silk and Hydrolyzed Silk PG-Propyl Methylsilanediol. It is furthermore possible to make use of the commercially available products with the INCI names Palmitoyl Oligopeptide, Palmitoyl Pentapeptide-3, Palmitoyl Pentapeptide-2, Acetyl Hexapeptide-1, Acetyl Hexapeptide-3, Copper Tripeptide-1, Hexapeptide-1, Hexapeptide-2, MEA-Hydrolyzed Silk.

The action of the active ingredient complex (A) may be further enhanced by the addition of fatty substances. Fatty substances should be taken to mean fatty acids, fatty alcohols, natural and synthetic waxes, which may assume both solid form and liquid form in an aqueous dispersion, and natural and synthetic cosmetic oil components.

Protein hydrolysates of plant origin, for example, soy, almond, pea, potato and wheat protein hydrolysates, are obtainable under the trademarks Gluadin® (Cognis), DiaMin® (Diamalt), Lexein® (Inolex), Hydrosoy® (Croda), Hydrolupin® (Croda), Hydrosesame® (Croda), Hydrotritium® (Croda) and Crotein® (Croda).

Although the use of protein hydrolysates as such is preferred, amino acid mixtures obtained in other ways may also optionally be used in their stead. It is likewise possible to use derivatives of protein hydrolysates, for example in the form of the fatty acid condensation products thereof. Such products are distributed for example under the names Lamepon® (Cognis), Lexein® (Inolex), Crolastin® (Croda), Crosilk® (Croda) or Crotein® (Croda).

It goes without saying that all isomeric forms, such as cis-trans isomers, diastereomers and chiral isomers are contemplated herein.

It is also possible to use a mixture of a plurality of protein hydrolysates.

In an exemplary embodiment, the protein hydrolysates are present in the agents, for example, in concentrations of about 0.01 wt. % up to about 20 wt. %, preferably of about 0.05 wt. % up to about 15 wt. % and more preferably of about 0.05 wt. % up to about 5 wt. %, in each case relative to the total ready-to-use preparation.

The agent may for example contain at least one cationic surfactant as a conditioner from another class of compounds.

In one embodiment, preference is given to cationic surfactants of the type including quaternary ammonium compounds, ester quats and amidoamines. Preferred quaternary ammonium compounds are ammonium halides, in particular chlorides and bromides, such as alkyltrimethylammonium chlorides, dialkyldimethylammonium chlorides and trialkylmethylammonium chlorides, for example cetyltrimethylammonium chloride, stearyltrimethylammonium chloride, distearyldimethylammonium chloride, lauryldimethylammonium chloride, lauryldimethylbenzylammonium chloride and tricetylmethylammonium chloride, and the imidazolinium compounds known under the INCI names Quaternium-27 and Quaternium-83. The long alkyl chains of the above-stated surfactants preferably comprise 10 to 18 carbon atoms.

Ester quats are known substances which contain both at least one ester function and at least one quaternary ammonium group as a structural element. Preferred ester quats are quaternized ester salts of fatty acids with triethanolamine, quaternized ester salts of fatty acids with diethanolalkylamines and quaternized ester salts of fatty acids with 1,2-dihydroxypropyldialkylamines. Such products are distributed, for example, under the trademarks Stepantex®, Dehyquart® and Armocare®. The products Armocare® VGH-70, an N,N-bis(2-palmitoyloxyethyl)dimethylammonium chloride, and Dehyquart® F-75, Dehyquart® C-4046, Dehyquart® L80 and Dehyquart® AU-35 are examples of such ester quats.

The alkylamidoamines are conventionally produced by amidating natural or synthetic fatty acids and fatty acid cuts with dialkylaminoamines. One compound from this group of substances which is particularly suitable is stearamidopropyldimethylamine, which is commercially available under the name Tegoamid® S 18.

In one embodiment, the agents contain the cationic surfactants in quantities of from about 0.05 to about 10 wt. %, relative to the total ready-to-use preparation. Quantities of about 0.1 to about 5 wt. % are preferred.

Conditioning polymers are likewise suitable as a conditioner. It should be noted at this point that some of the conditioning polymers stated below also exhibit film-forming and/or setting properties and so may also be found in the description of suitable film-forming and/or setting polymers.

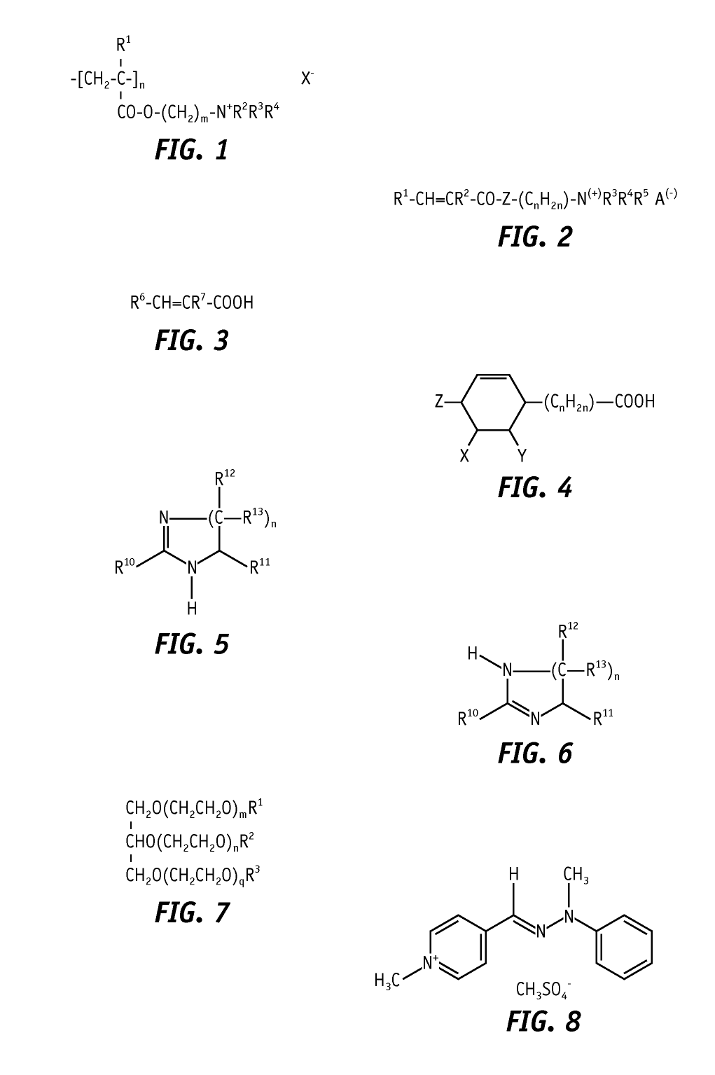

A first group of conditioning polymers comprises cationic polymers. Cationic polymers are polymers which comprise a group in the main and/or side chain which may be “temporarily” or “permanently” cationic. Polymers which are designated “permanently cationic” are those which, irrespective of the pH value of the agent, comprise a cationic group. As a rule, these are polymers which contain a quaternary nitrogen atom, for example in the form of an ammonium group. Preferred cationic groups are quaternary ammonium groups. Polymers which have proven particularly suitable are those in which the quaternary ammonium group is bound via a C₁₋₄ hydrocarbon group to a main polymer chain synthesized from acrylic acid, methacrylic acid or the derivatives thereof. Homopolymers of the general formula of FIG. 1 in which R¹ is —H or —CH₃, R², R³ and R⁴ are mutually independently selected from C₁₋₄ alkyl, alkenyl or hydroxyalkyl groups, m=1, 2, 3 or 4, n is a natural number, and X⁻ a physiologically acceptable organic or inorganic anion, and copolymers substantially consisting of the monomer units listed in the formula of FIG. 1 and nonionogenic monomer units, are preferred cationic polymers. In the context of these polymers, those which are more preferred are those for which at least one of the following conditions applies:

R¹ denotes a methyl group;
R², R³ and R⁴ denote methyl groups; and
m has the value 2.

Physiologically acceptable counterions X⁻ which may, for example, be considered are halide ions, sulfate ions, phosphate ions, methosulfate ions and organic ions such as lactate, citrate, tartrate and acetate ions. Halide ions, in particular chloride, are preferred.

A particularly suitable homopolymer is poly(methacryloyloxyethyl-trimethylammonium chloride), which may if desired be crosslinked, with the INCI name of Polyquaternium-37. Crosslinking may if desired proceed with the assistance of olefinically polyunsaturated compounds, for example divinylbenzene, tetraallyloxyethane, methylenebisacrylamide, diallyl ether, polyallyl polyglyceryl ether, or allyl ethers of sugars or sugar derivatives such as erythritol, pentaerythritol, arabitol, mannitol, sorbitol, sucrose or glucose. Methylenebisacrylamide is a preferred crosslinking agent.

The homopolymer is preferably used in the form of a nonaqueous polymer dispersion which should have a polymer fraction of no less than about 30 wt. %. Such polymer dispersions are commercially available under the names Salcare® SC 95 (approximately 50% polymer fraction, further components: mineral oil (INCI name: Mineral Oil) and tridecyl-polyoxypropylene-polyoxyethylene ether (INCI name: PPG-1-Trideceth-6)) and Salcare® SC 96 (approx. 50% polymer fraction, further components: mixture of diesters of propylene glycol with a mixture of caprylic and capric acid (INCI name: Propylene Glycol Dicaprylate/Dicaprate) and tridecyl-polyoxypropylene-polyoxyethylene ether (INCI name: PPG-1-Trideceth-6)).

Copolymers with monomer units according to the formula of FIG. 1 preferably contain acrylamide, methacrylamide, acrylic acid C₁₋₄ alkyl esters and methacrylic acid C₁₋₄ alkyl esters as nonionogenic monomer units. Acrylamide is particularly preferred among these nonionogenic monomers. These copolymers, as described above for the homopolymers, may also be crosslinked. A copolymer which is preferred according to the invention is crosslinked acrylamide-methacryloyloxyethyltrimethylammonium chloride copolymer. Such copolymers, in which the monomers are present in a weight ratio of about 20:80, are commercially available as about 50% nonaqueous polymer dispersions under the name Salcare® SC 92.

Further preferred cationic polymers are, for example:
quaternized cellulose derivatives, as are commercially available under the names Celquat® and Polymer JR®. The compounds Celquat® H 100, Celquat® L 200 and polymer JR®400 are preferred quaternized cellulose derivatives;
cationic alkyl polyglycosides according to German patent DE-PS 44 13 686;
cationized honey, for example the commercial product Honeyquat® 50;
cationic guar derivatives, such as, in particular, the products distributed under the trade names Cosmedia®Guar and Jaguar;
polysiloxanes with quaternary groups, such as for example the commercially obtainable products Q2-7224 (manufacturer: Dow Corning Corporation of Midland Mich.; a stabilized trimethylsilylamodimethicone), Dow Corning® 929 Emulsion (containing a hydroxylamine-modified silicone which is also designated an amodimethicone), SM-2059 (manufacturer: General Electric), SLM-55067 (manufacturer: Wacker) and Abil®-Quat 3270 and 3272 (manufacturer: Th. Goldschmidt; diquaternary polydimethylsiloxane, Quaternium-80);
polymeric dimethyldiallylammonium salts and the copolymers thereof with esters and amides of acrylic acid and methacrylic acid. The products commercially available under the names Merquat®100 (poly(dimethyldiallylammonium chloride)) and Merquat®550 (dimethyldiallylammonium chloride/acrylamide copolymer) are examples of such cationic polymers;
copolymers of vinylpyrrolidone with quaternized derivatives of dialkylaminoalkyl acrylate and methacrylate, such as for example vinylpyrrolidone/dimethylaminoethyl methacrylate copolymers quaternized with diethyl sulfate. Such compounds are commercially available under the names Gafquat®734 and Gafquat®755; vinylpyrrolidone/vinylimidazolium methochloride copolymers, as are offered for sale under the names Luviquat® FC 370, FC 550, FC 905 and HM 552;
quaternized polyvinyl alcohol; and
and the polymers known by the names Polyquaternium 2, Polyquaternium 17, Polyquaternium 18 and Polyquaternium 27 with quaternary nitrogen atoms in the polymer main chain.

The polymers known under the names Polyquaternium-24 (commercial product, for example Quatrisoft® LM 200) may also be used as cationic polymers. Copolymers of vinylpyrrolidone, as are available as commercial products Copolymer 845 (manufacturer: ISP), Gaffix® VC 713 (manufacturer: ISP), Gafquat®ASCP 1011, Gafquat®HS 110, Luviquat®8155 and Luviquat® MS 370 may likewise be used.

Further cationic polymers which are usable are “temporarily cationic” polymers. These polymers conventionally contain an amino group which at specific pH values assumes the form of a quaternary ammonium group and is thus cationic. Chitosan and the derivatives thereof are for example preferred, as are those readily commercially available for example under the trade names Hydagen® CMF, Hydagen® HCMF, Kytamer® PC and Chitolam® NB/101.

Cationic polymers which are preferably used are cationic cellulose derivatives and chitosan and the derivatives thereof, in particular the commercial products Polymer®JR 400, Hydagen® HCMF and Kytamer® PC, cationic guar derivatives, cationic honey derivatives, in particular the commercial product Honeyquat® 50, cationic alkyl polyglycosides according to German patent DE-PS 44 13 686 and polymers of the Polyquaternium-37 type.

Cationic polymers additionally include protein hydrolysates, the underlying protein hydrolysate possibly originating from animals, for example from collagen, milk or keratin, from plants, for example from wheat, maize, rice, potatoes, soy or almonds, from marine life forms, for example from fish collagen or algae, or biotechnologically obtained protein hydrolysates. The protein hydrolysates underlying the cationic derivatives may be obtained from the corresponding proteins by chemical, in particular alkaline or acidic, hydrolysis, by enzymatic hydrolysis and/or by a combination of both types of hydrolysis. Protein hydrolysis as a rule gives rise to a protein hydrolysate with a molecular weight distribution of approximately 100 daltons up to several thousand daltons. Those cationic protein hydrolysates are preferred whose underlying protein fraction has a molecular weight of about 100 up to about 25,000 daltons, preferably about 250 to about 5,000 daltons. Furthermore, cationic protein hydrolysates include quaternized amino acids and mixtures thereof. Quaternization of the protein hydrolysates or of the amino acids is often performed by means of quaternary ammonium salts such as for example N,N-dimethyl-N-(n-alkyl)-N-(2-hydroxy-3-chloro-n-propyl)-ammonium halides. The cationic protein hydrolysates may additionally be still further derivatized. Typical examples of the cationic protein hydrolysates and derivatives according to the invention which may be mentioned are those that are commercially obtainable and mentioned under the INCI names in the “International Cosmetic Ingredient Dictionary and Handbook”, (seventh edition 1997, The Cosmetic, Toiletry, and Fragrance Association 1101 17th Street, N.W., Suite 300, Washington, D.C. 20036-4702): Cocodimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Soy Protein, Cocodimonium Hydroxypropyl Hydrolyzed Wheat Protein, Hydroxypropyl Arginine Lauryl/Myristyl Ether HCl, Hydroxypropyl-trimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed Keratin, Hydroxypropyltrimonium Hydrolyzed Rice Bran Protein, Hydroxypropyl-trimonium Hydrolyzed Soy Protein, Hydroxypropyl Hydrolyzed Vegetable Protein, Hydroxypropyltrimonium Hydrolyzed Wheat Protein, Hydroxypropyl-trimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Soy Protein, and Quaternium-79 Hydrolyzed Wheat Protein.

Plant-based cationic protein hydrolysates and derivatives are very particularly preferred.

Amphoteric polymers which are preferably used are those polymers which are substantially composed of:
(a) monomers with quaternary ammonium groups of the general formula of FIG. 2;
   in which R¹ and R² mutually independently denote hydrogen or a methyl group and R³, R⁴ and R⁵ mutually independently denote alkyl groups having 1 to 4 carbon atoms, Z is an NH group or an oxygen atom, n is an integer from 2 to 5 and A⁽⁻⁾ is the anion of an organic or inorganic acid; and
(b) monomeric carboxylic acids of the general formula of FIG. 3,
   in which R⁶ and R⁷ mutually independently denote hydrogen or a methyl group.

These compounds may be used both directly and in salt form, which is obtained by neutralization of the polymers, for example with an alkali metal hydroxide. Preferred polymers are those in which monomers of type (a) are used, in which R³, R⁴ and R⁵ are methyl groups, Z is an NH group and A⁽⁻⁾ is a halide, methoxysulfate or ethoxysulfate ion; acrylamidopropyltrimethylammonium chloride is a particularly preferred monomer (a). Acrylic acid is preferably used as monomer (b) for the stated polymers.

The agents preferably contain the conditioning, cationic or amphoteric polymers in a quantity of about 0.01 to about 5 wt. %, more preferably in a quantity of about 0.1 to about 2 wt. %, in each case relative to the total ready-to-use preparation.

The agent may furthermore contain at least one vitamin, a provitamin, a vitamin precursor and/or one of the derivatives thereof as a conditioner.

The vitamins, provitamins and vitamin precursors which are preferred are those which are conventionally assigned to the groups A, B, C, E, F and H.

The group of substances designated vitamin A includes retinol (vitamin A₁) and 3,4-didehydroretinol (vitamin A₂). β-Carotene is the provitamin of retinol. Examples of substances which may be considered according the invention as the vitamin A component are vitamin A acid and the esters thereof, vitamin A aldehyde and vitamin A alcohol and the esters thereof such as the palmitate and the acetate. The agents preferably contain the vitamin A component in quantities of from about 0.05 to about 1 wt. %, relative to the total ready-to-use preparation.

The vitamin B group or the vitamin B complex includes, inter alia:
vitamin B₁ (thiamin);
vitamin B₂ (riboflavine);
vitamin B₃. This designation is frequently used for the compounds nicotinic acid and nicotinamide (niacinamide). In one embodiment, nicotinamide is contained in the agents in quantities of from about 0.05 to about 1 wt. %, relative to the total ready-to-use preparation;
Vitamin B₅ (pantothenic acid, panthenol and pantolactone). In the context of this group, panthenol and/or pantolactone are preferably used. Derivatives of panthenol which may be used are in particular the esters and ethers of panthenol and cationically derivatized panthenols. Individual representatives are for example panthenol triacetate, the panthenol monoethyl ether and the monoacetate thereof and the panthenol derivatives disclosed in WO 92/13829. The stated compounds of the vitamin B₅ type are preferably contained in the agents in quantities of from about 0.05 to about 10 wt. %, relative to the total agent. Quantities of about 0.1 to about 5 wt. % are particularly preferred; and
Vitamin B₆ (pyridoxine as well as pyridoxamine and pyridoxal). In a preferred embodiment, the stated compounds of the vitamin B₆ type are contained in the agents in quantities of from about 0.01 to about 5 wt. %, relative to the total agent. Quantities of about 0.05 to about 1 wt. % are particularly preferred.

Vitamin C (ascorbic acid). Vitamin C is preferably used in the agents in quantities of from about 0.1 to about 3 wt. %, relative to the total agent. Use in the form of the palmitic acid ester, the glucosides or phosphates may be preferred. Use in combination with tocopherols may likewise be preferred.

Vitamin E (tocopherols, in particular α-tocopherol). Tocopherol and the derivatives thereof, which include in particular the esters such as the acetate, the nicotinate, the phosphate and the succinate, are preferably contained in the agents in quantities of from about 0.05 to about 1 wt. %, relative to the total agent.

Vitamin F. The term “vitamin F” is conventionally understood to mean essential fatty acids, in particular linoleic acid, linolenic acid and arachidonic acid.

Vitamin H. Vitamin H denotes the compound (3aS,4S,6aR)-2-oxohexahydrothienol[3,4-d]-imidazole-4-valeric acid, which is now known however by the common name biotin. In an exemplary embodiment, biotin is contained in the agents in quantities of from about 0.0001 to about 1.0 wt. %, preferrably in quantities of from about 0.001 to about 0.01 wt %, relative to the total ready-to-use preparation.

The agents contemplated herein preferably contain vitamins, provitamins and vitamin precursors from groups A, B, C, E and H.

Panthenol, pantolactone, pyridoxine and the derivatives thereof together with nicotinamide and biotin are particularly preferred.

D-Panthenol is very particularly preferably used as a conditioner from this group.

The agents may furthermore contain at least one plant extract as conditioner.

Conventionally, these extracts are produced by extraction of the entire plant. However, in individual cases it may also be preferable to produce the extracts solely from the blossoms and/or leaves of the plant.

With regard to preferred plant extracts, reference is made in particular to the extracts which are listed in the table starting on page 44 of the 3rd edition of the “Leitfaden zur Inhaltsstoffdeklaration kosmetischer Mittel” [“Guidelines for the nomenclature of ingredients in cosmetic agents”], published by the German Cosmetic, Toiletry, Perfumery and Detergent Association (IKW), Frankfurt, Germany.

Preference is given to extracts from green tea, oak bark, stinging nettle, witch hazel, hops, henna, chamomile, burdock root, horsetail, hawthorn, lime blossom, almond, aloe vera, pine-needle, horse chestnut, sandalwood, juniper, coconut, mango, apricot, lime, wheat, kiwi fruit, melon, orange, grapefruit, sage, rosemary, birch, mallow, lady's smock, wild thyme, yarrow, thyme, melissa, restharrow, coltsfoot, marsh mallow, meristem, ginseng and ginger root.

Particular preference is given to the extracts of green tea, oak bark, stinging nettle, witch hazel, hops, chamomile, burdock root, horsetail, lime blossom, almond, aloe vera, coconut, mango, apricot, lime, wheat, kiwi fruit, melon, orange, grapefruit, sage, rosemary, birch, lady's smock, wild thyme, yarrow, restharrow, meristem, ginseng and ginger root.

The extracts of green tea, almond, aloe vera, coconut, mango, apricot, lime, wheat, kiwi fruit and melon are very particularly suitable.

Extracting agents for producing the stated plant extracts may comprise water, alcohols and mixtures thereof. Preferred alcohols are lower alcohols such as ethanol and isopropanol, but in particular polyhydric alcohols such as ethylene glycol and propylene glycol, both as sole extracting agent and in a mixture with water. Plant extracts based on water/propylene glycol in the ratio of about 1:10 to about 10:1 have proven particularly suitable.

The plant extracts may be used both in pure and in dilute form. Where used in dilute form, they conventionally contain approximately 2-80 wt. % of active substance and contain as a solvent the extracting agent or extracting agent mixture used to isolate them.

It may furthermore be preferred to use mixtures of a plurality of, in particular of two, different plant extracts in the agents contemplated herein.

Also suitable as conditioner are a series of carboxylic acids.

Short-chain carboxylic acids in particular may be advantageous for use in the agents contemplated herein. As used herein, short-chain carboxylic acids and the derivatives thereof are understood to mean carboxylic acids which may be saturated or unsaturated and/or linear or branched or cyclic and/or aromatic and/or heterocyclic and have a molecular weight of less than 750. In a preferred embodiment, saturated or unsaturated linear or branched carboxylic acids with a chain length of from 1 up to 16 C atoms in the chain are used. Those with a chain length of from 1 up to 12 C atoms are more preferred.

The short-chain carboxylic acids may comprise one, two, three or more carboxy groups. Carboxylic acids having a plurality of carboxy groups, in particular di- and tricarboxylic acids, are preferred. The carboxy groups may be present entirely or in part as esters, acid anhydride, lactone, amide, imidic acid, lactam, lactim, dicarboximide, carbohydrazide, hydrazone, hydroxam, hydroxime, amidine, amide oxime, nitrile, phosphonate or phosphate esters. It goes without saying that the carboxylic acids may be substituted along the carbon chain or the ring skeleton. The substituents of carboxylic acids include for example C₁-C₈ alkyl, C₂-C₈ alkenyl, aryl, aralkyl and aralkenyl, hydroxymethyl, C₂-C₈ hydroxyalkyl, C₂-C₈ hydroxyalkenyl, aminomethyl, C₂-C₈ aminoalkyl, cyano, formyl, oxo, thioxo, hydroxy, mercapto, amino, carboxy or imino groups. Preferred substituents are C₁-C₈ alkyl, hydroxymethyl, hydroxy, amino and carboxy groups. Substituents in a position are very particularly preferred. Very particularly preferred substituents are hydroxy, alkoxy and amino groups, the amino function optionally being further substituted by alkyl, aryl, aralkyl and/or alkenyl residues. Phosphonate and phosphate esters are furthermore likewise preferred carboxylic acid derivatives.

Examples of carboxylic acids suitable for use in the agents are formic acid, acetic acid, propionic acid, butyric acid, isobutyric acid, valeric acid, isovaleric acid, pivalic acid, oxalic acid, malonic acid, succinic acid, glutaric acid, glyceric acid, glyoxylic acid, adipic acid, pimelic acid, suberic acid, azelaic acid, sebacic acid, propiolic acid, crotonic acid, isocrotonic acid, elaidic acid, maleic acid, fumaric acid, muconic acid, citraconic acid, mesaconic acid, camphoric acid, benzoic acid, o,m,p-phthalic acid, naphthoic acid, toluic acid, hydratropic acid, atropic acid, cinnamic acid, isonicotinic acid, nicotinic acid, bicarbamic acid, 4,4′-dicyano-6,6′-binicotinic acid, 8-carbamoyloctanoic acid, 1,2,4-pentanetricarboxylic acid, 2-pyrrolecarboxylic acid, 1,2,4,6,7-naphthalenepentaacetic acid, malonaldehydic acid, 4-hydroxyphthalamidic acid, 1-pyrazolecarboxyilic acid, gallic acid or propanetricarboxylic acid, a dicarboxylic acid selected from the group which is formed by compounds of the general formula of FIG. 4,

in which Z denotes a linear or branched, alkyl or alkenyl group with 4 to 12 carbon atoms, n denotes a number from 4 to 12 and one of the two groups X and Y denotes a COOH group and the other denotes hydrogen or a methyl or ethyl residue, dicarboxylic acids of the general formula of FIG. 4, which additionally also bear 1 to 3 methyl or ethyl substituents on the cyclohexene ring and dicarboxylic acids formally arising from the dicarboxylic acids of FIG. 4 by attachment of a molecule of water onto the double bond in the cyclohexene ring.

Dicarboxylic acids of the formula of FIG. 4 are known in the literature. A production method is disclosed, for example, in U.S. Pat. No. 3,753,968.

The dicarboxylic acids of the formula of FIG. 4 may be produced, for example, by reacting polyunsaturated dicarboxylic acids with unsaturated monocarboxylic acids in the manner of a Diels-Alder cyclization. A polyunsaturated fatty acid will conventionally be used as starting material for a dicarboxylic acid component. Linoleic acid, which is obtainable from natural fats and oils, is preferred. Acrylic acid, but also for example methacrylic acid and crotonic acid, are in particular preferred as the monocarboxylic acid component. Diels-Alder reactions conventionally give rise to isomer mixtures in which one component is present in excess. These isomer mixtures may likewise be used in the same manner as the pure compounds.

In addition to the preferred dicarboxylic acids according to the formula of FIG. 4, it is also possible to use those dicarboxylic acids which differ from the compounds according to the formula of FIG. 4 by 1 to 3 methyl or ethyl substituents on the cyclohexyl ring or are formally formed from these compounds by attachment of a molecule of water onto the double bond of the cyclohexene ring.

The dicarboxylic acid (mixture) obtained by reacting linoleic acid with acrylic acid has proved particularly active. This is a mixture of 5- and 6-carboxy-4-hexyl-2-cyclohexene-1-octanoic acid. Such compounds are commercially available under the names Westvaco Diacid® 1550 and Westvaco Diacid® 1595 (manufacturer: Meadwestvaco Corporation of Glen Allen, Va.).

In addition to the short-chain carboxylic acids listed above by way of example themselves, the physiologically acceptable salts thereof may also be used in accordance with an exemplary embodiment. Examples of such salts are the alkali metal, alkaline earth metal, zinc salts and ammonium salts, these being understood within the bounds of present application also to mean the mono-, di- and trimethyl-, -ethyl- and -hydroxyethylammonium salts. However, for the purposes herein, neutralized acids may preferably be used with alkaline-reacting amino acids, such as for example arginine, lysine, ornithine and histidine. It may furthermore be preferred for formulation reasons to select the carboxylic acids from among the water-soluble representatives, in particular the water-soluble salts.

It is furthermore preferred according to the invention to use 2-pyrrolidinone-5-carboxylic acid and the derivatives thereof as the carboxylic acid. The sodium, potassium, calcium, magnesium or ammonium salts, in which, in addition to hydrogen, the ammonium ion bears one to three C₁ to C₄ alkyl groups, are particularly preferred. The sodium salt is very particularly preferred. The agents contemplated herein use from about 0.05 to about 10 wt. %, relative to the total ready-to-use preparation, preferably from about 0.1 to about 5 wt. %, and more preferably from about 0.1 to about 3 wt. %.

It is furthermore preferred in an exemplary embodiment to use hydroxycarboxylic acids and in turn in particular the dihydroxy-, trihydroxy- and polyhydroxycarboxylic acids and the dihydroxy-, trihydroxy- and polyhydroxy-di-, tri- and polycarboxylic acids. It has here been found that, in addition to hydroxycarboxylic acids, the hydroxycarboxylic acid esters and mixtures of hydroxycarboxylic acids and the esters thereof as well as polymeric hydroxycarboxylic acids and the esters thereof may be very particularly preferred. Preferred hydroxycarboxylic acid esters are for example full esters of glycolic acid, lactic acid, malic acid, tartaric acid or citric acid. Further hydroxycarboxylic acid esters which are suitable in principle are esters of β-hydroxypropionic acid, of tartronic acid, of D-gluconic acid, of saccharic acid, of mucic acid or of glucuronic acid. Suitable alcohol components of these esters are primary, linear or branched aliphatic alcohols having 8-22 C atoms, thus for example fatty alcohols or synthetic fatty alcohols. The esters of C₁₂-C₁₅ fatty alcohols are here particularly preferred. Esters of this type are commercially available, for example under the trademark Cosmacol® from EniChem, Augusta Industriale of Italy. Particularly preferred polyhydroxypolycarboxylic acids are polylactic acid and polytartaric acid and the esters thereof.

Ectoin, or ectoin derivatives, allantoin, taurine and/or bisabolol are furthermore suitable as conditioner.

As used herein, “ectoin and ectoin derivatives” are taken to mean compounds of the formulas of FIG. 5 and FIG. 6,

and/or the physiologically acceptable salts and/or isomeric or stereoisomeric forms thereof, in which:
R¹⁰ denotes a hydrogen atom, a branched or unbranched C₁-C₄ alkyl residue or a C₂-C₄ hydroxyalkyl residue;
R¹¹ denotes a hydrogen atom, a —COOR¹⁴ grouping or a —CO(NH)R¹⁴ grouping, with R¹⁴ possibly denoting a hydrogen atom, a C₁-C₄ alkyl residue, an amino acid residue, a dipeptide or tripeptide residue'
R¹² and R¹³ mutually independently denote a hydrogen atom, a C₁-C₄ alkyl residue or a hydroxy group, with the proviso that both residues must not
simultaneously denote a hydroxy group; and
n denotes an integer from 1 to 3.

Suitable physiologically acceptable salts of the general compounds of the formulas of FIG. 5 or FIG. 6 are for example the alkali metal, alkaline earth metal, ammonium, triethylamine or tris-(2-hydroxyethyl)amine salts and the like, which arise from the reaction of compounds of the formulas of FIG. 5 or FIG. 6 with inorganic and organic acids such as hydrochloric acid, phosphoric acid, sulfuric acid, branched or unbranched, substituted or unsubstituted (for example by one or more hydroxy groups) C₁-C₄ mono- or dicarboxylic acids, aromatic carboxylic acids and sulfonic acids such as acetic acid, citric acid, benzoic acid, maleic acid, fumaric acid, tartaric acid and p-toluenesulfonic acid. Examples of particularly preferred physiologically acceptable salts are the Na, K, Mg and Ca and ammonium salts of the compounds of the formulas of FIG. 5 or FIG. 6, and the salts which arise by reacting compounds of the formulas of FIG. 5 or FIG. 6 with hydrochloric acid, acetic acid, citric acid and benzoic acid.

Isomeric or stereoisomeric forms of the compounds of FIG. 5 or FIG. 6 are taken herein to mean all occurring optical isomers, diastereomers, racemates, zwitterions, cations or mixtures thereof.

The term amino acid is taken to mean the stereoisomeric forms, for example D- and L-forms, of the following compounds:

asparagine, arginine, aspartic acid, glutamine, glutamic acid, β-alanine, γ-aminobutyrate, N_{ε}-acetyllysine, N_{δ}-acetylornithine, N_{γ}-acetyldiaminobutyrate, N_{α}-acetyldiaminobutyrate, histidine, isoleucine, leucine, methionine, phenyl-alanine, serine, threonine and tyrosine.
L-amino acids are preferred. Amino acid residues are derived from the corresponding amino acids. The following amino acid residues are preferred:
Gly, Ala, Ser, Thr, Val, β-Ala, γ-aminobutyrate, Asp, Glu, Asn, Aln, N_{ε}-acetyllysine, N_{δ}-acetylornithine, N_{γ}-acetyldiaminobutyrate, N_{α} acetyldiamino-butyrate.

The amino acids have been abbreviated in line with the usual spelling. Chemically, the di- or tripeptide residues are acid amides and, on hydrolysis, break down into 2 or 3 amino acids. The amino acids in the di- or tripeptide residue are joined to one another by amide bonds.

Explicit reference is made to European patent EP 0 671 161 A1 (Marbert) with regard to production of the di- and tripeptide residues. Examples of di- and tripeptide residues may also be found in the disclosure of European patent EP 0 671 161 A1.

Examples of C₁-C₄ alkyl groups in the compounds of the formulas of FIG. 5 or FIG. 6 are methyl, ethyl, n-propyl, isopropyl, n-butyl, isobutyl and tert.-butyl. Preferred alkyl groups are methyl and ethyl; methyl is a particularly preferred alkyl group. Preferred C₂-C₄ hydroxyalkyl groups are the groups 2-hydroxyethyl, 3-hydroxypropyl or 4-hydroxybutyl; 2-hydroxyethyl is a particularly preferred hydroxyalkyl group.

In a preferred embodiment, the agents contain these conditioners in quantities of from about 0.001 to about 2, in particular of from about 0.01 to about 0.5 wt. %, in each case relative to the total ready-to-use preparation.

Mono- or oligosaccharides may also be used as a conditioner in the agents.

Both monosaccharides and oligosaccharides, such as for example cane sugar, lactose and raffinose, may be used. The use of monosaccharides is preferred. Among the monosaccharides, those compounds containing 5 or 6 carbon atoms are in turn preferred.

Suitable pentoses and hexoses are for example ribose, arabinose, xylose, lyxose, allose, altrose, glucose, mannose, gulose, idose, galactose, talose, fucose and fructose. Arabinose, glucose, galactose and fructose are preferably used carbohydrates; glucose, which is suitable either in D-(+)- or L-(−)-configuration or as a racemate, is very particularly preferably used.

Derivatives of these pentoses and hexoses, such as the corresponding -onic and -uronic acids (saccharic acids), sugar alcohols and glycosides, may furthermore be used. Preferred saccharic acids are gluconic acid, glucuronic acid, saccharic acid, mannosaccharic acid and mucic acid. Preferred sugar alcohols are sorbitol, mannitol and dulcitol. Preferred glycosides are the methyl glucosides.

Since the mono- or oligosaccharides used are conventionally obtained from natural raw materials such as starch, they generally exhibit the configurations corresponding to these raw materials (for example D-glucose, D-fructose and D-galactose).

In an exemplary embodiment, the mono- or oligosaccharides are present in the hair treatment agents in a quantity of from about 0.1 to about 8 wt. %, preferably from about 1 to about 5 wt. %, relative to the total ready-to-use preparation.

The agent may furthermore contain at least one lipid as conditioner.

Lipids which are suitable for use in the agents are phospholipids, for example soy lecithin, egg lecithin and cephalins and the substances known under the INCI names Linoleamidopropyl PG-Dimonium Chloride Phosphate, Cocamidopropyl PG-Dimonium Chloride Phosphate and Stearamidopropyl PG-Dimonium Chloride Phosphate. These are distributed for example by Mona under the trade names Phospholipid EFA®, Phospholipid PTC® and Phospholipid SV®.

In a preferred embodiment, agents contain the lipids in quantities of from about 0.01 to about 10 wt. %, more preferrably of from about 0.1 to about 5 wt. %, relative to the total ready-to-use preparation.

Oil bodies are furthermore suitable as a conditioner.

Natural and synthetic cosmetic oil bodies include, for example:
vegetable oils. Examples of such oils are sunflower oil, olive oil, soy oil, rapeseed oil, almond oil, jojoba oil, orange oil, wheat germ oil, peach stone oil and the liquid fractions of coconut oil. However, other triglyceride oils such as the liquid fractions of beef fat together with synthetic triglyceride oils are also suitable;
liquid paraffin oils, isoparaffin oils and synthetic hydrocarbons and di-n-alkyl ethers having a total of between 12 to 36 C atoms, in particular 12 to 24 C atoms, such as for example di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether and di-tert.-butyl ether, di-iso-pentyl ether, di-3-ethyldecyl ether, tert.-butyl-n-octyl ether, iso-pentyl-n-octyl ether and 2-methylpentyl-n-octyl ether. The compounds 1,3-di-(2-ethylhexyl)cyclohexane (Cetiol® S) and di-n-octyl ether (Cetiol®OE) available as commercial products may be preferred;
ester oils. Ester oils should be taken to mean the esters of C₆-C₃₀ fatty acids with C₂-C₃₀ fatty alcohols. The monoesters of fatty acids with alcohols having 2 to 24 C atoms are preferred. Examples of fatty acid moieties used in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid and erucic acid and the technical mixtures thereof which are obtained, for example, on pressure splitting of natural fats and oils, on oxidation of aldehydes from Roelen's oxo synthesis or on dimerization of unsaturated fatty acids. Examples of fatty alcohol moieties in the ester oils are isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and the technical mixtures thereof, which are obtained, for example, on high pressure hydrogenation of technical methyl esters based on fats and oils or aldehydes from Roelen's oxo synthesis and as the monomer fraction on dimerization of unsaturated fatty alcohols. Particularly preferred substances are isopropyl myristate (Rilanit® IPM), isononanoic acid C16-18 alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® mm), cetearyl isononanoate (Cetiol® SN), oleic acid decyl ester (Cetiol® V);
dicarboxylic acid esters such as di-n-butyl adipate, di-(2-ethylhexyl) adipate, di-(2-ethylhexyl) succinate and diisotridecyl acelate and diol esters such as ethylene glycol dioleate, ethylene glycol diisotridecanoate, propylene glycol di-(2-ethylhexanoate), propylene glycol diisostearate, propylene glycol dipelargonate, butanediol diisostearate, neopentyl glycol dicaprylate;
symmetrical, asymmetrical or cyclic esters of carbonic acid with fatty alcohols, for example described in German patent DE-OS 197 56 454, glycerol carbonate or dicaprylyl carbonate (Cetiol® CC);
trifatty acid esters of saturated and/or unsaturated linear and/or branched fatty acids with glycerol;
fatty acid partial glycerides, which are taken to mean monoglycerides, diglycerides and the technical mixtures thereof. When using technical products, small quantities of triglycerides may still be contained therein, depending on the production method. Partial glycerides preferably follow the formula of FIG. 7,
in which R¹, R² and R³ mutually independently denote hydrogen or a linear or branched, saturated and/or unsaturated acyl residue with 6 to 22, preferably 12 to 18, carbon atoms, with the proviso that at least one of these groups denotes an acyl residue and at least one of these groups denotes hydrogen and the sum (m+n+q) denotes 0 or numbers from 1 to 100, preferably 0 or 5 to 25. Preferably, R¹ denotes an acyl residue and R² and R³ denote hydrogen and the sum (m+n+q) is 0. Typical examples are mono- and/or diglycerides based on caproic acid, caprylic acid, 2-ethylhexanoic acid, capric acid, lauric acid, isotridecanoic acid, myristic acid, palmitic acid, palmoleic acid, stearic acid, isostearic acid, oleic acid, elaidic acid, petroselinic acid, linoleic acid, linolenic acid, elaeostearic acid, arachidic acid, gadoleic acid, behenic acid and erucic acid and the technical mixtures thereof. Preferably, oleic acid monoglycerides are used.

In an exemplary embodiment, the input quantity of the natural and synthetic cosmetic oil bodies in the agents is in the range of from about 0.1 to about 30 wt. % relative to the total ready-to-use preparation, preferably from about 0.1 to about 20 wt. %, and more preferably from about 0.1 to about 15 wt. %.

The agents may moreover contain an enzyme as conditioner. Enzymes which are particularly preferred are selected from a group which is formed of proteases, lipases, transglutaminase, oxidases and peroxidases.

Pearl extracts are also suitable as a conditioner.

Mussel pearls substantially consist of inorganic and organic calcium salts, trace elements and proteins. Pearls may straightforwardly be obtained from cultured mussels. Mussels can be cultured in both fresh water and seawater. This may have an impact on the constituents of the pearls. A pearl extract which is preferred is one which originates from mussels cultured in seawater or salt water. The pearls consist to a great extent of aragonite (calcium carbonate), conchiolin and an albuminoid. The latter constituents are proteins. Pearls furthermore additionally contain magnesium and sodium salts, inorganic silicon compounds and phosphates.

Pearl extract is produced by pulverizing the pearls. The pulverized pearls are then extracted using conventional methods. Extracting agents for producing the pearl extracts may comprise water, alcohols and mixtures thereof. Water should here be taken to mean both demineralized water and seawater. Preferred alcohols are lower alcohols such as ethanol and isopropanol, but in particular polyhydric alcohols such as glycerol, diglycerol, triglycerol, polyglycerol, ethylene glycol, propylene glycol and butylene glycol, both as sole extracting agent and in a mixture with demineralized water or seawater. Pearl extracts based on water/glycerol mixtures have proved particularly suitable. Depending on the extraction conditions, the pearl proteins (conchiolin and albuminoid) may be present very largely in their native state or already partially or very largely as protein hydrolysates. A preferred pearl extract is one in which conchiolin and albuminoid already assume partially hydrolysed form. The substantial amino acids of these proteins are glutamic acid, serine, alanine, glycine, aspartic acid and phenylalanine. In a more preferred embodiment, it may be advantageous for the pearl extract additionally to be enriched with at least one or more of these amino acids. In a most preferred embodiment, the pearl extract is enriched with glutamic acid, serine and leucine. Furthermore, depending on the extraction conditions, in particular depending on the extracting agent selected, the extract contains a greater or lesser proportion of minerals and trace elements. One preferred extract contains organic and/or inorganic calcium salts and magnesium and sodium salts, inorganic silicon compounds and/or phosphates. In one embodiment, the pearl extract contains at least about 75%, preferably about 85%, more preferably about 90% and most preferably about 95% of all the constituents of the naturally occurring pearls. Examples of pearl extracts usable according to the invention are the commercial products Pearl Protein Extract BG® or Crodarom® Pearl.

In an exemplary embodiment, the above-described pearl extracts are preferably present in a quantity of from at least about 0.01 up to about 20 wt. %. The quantities used of the extract are preferably from about 0.01 up to about 10 wt. %, and more preferably from about 0.01 to about 5 wt. % relative to the total agent.

Although each of the stated conditioners itself alone gives rise to a satisfactory result, in various embodiments contemplated herein the agent contains a plurality of conditioners, including from different groups.

Some of the stated conditioners, for example the above-stated oil bodies, may reduce adhesive strength, thus in particular surface tackiness (tack) and adhesion, of the pressure-sensitive adhesives used. The conditioners and also the further ingredients of the cosmetics must accordingly be carefully tailored to the pressure-sensitive adhesives used. The nature and concentration of these constituents are selected such that the adhesive strength of the pressure-sensitive is still at the desired level in the finished agent. Suitable combinations may readily be identified by simple preliminary testing.

In an exemplary embodiment, the agent is present in a cosmetically acceptable carrier. This preferably comprises an aqueous, an alcoholic or an aqueous/alcoholic medium preferably containing at least 10 weight percent of water relative to the total preparation. Alcohols which may be present are in particular the lower alcohols with 1 to 4 carbon atoms such as for example ethanol and isopropanol which are conventionally used for cosmetic purposes.

Additional cosolvents which may be present are organic solvents or a mixture of solvents with a boiling point of below 400° C. in a quantity of from about 0.1 to about 15 wt %, preferably of from about 1 to about 10 wt % relative to the total preparation. Particularly suitable additional cosolvents are unbranched or branched hydrocarbons such as pentane, hexane, isopentane and cyclic hydrocarbons such as cyclopentane and cyclohexane. Further, particularly preferred water-soluble solvents are glycerol, ethylene glycol, diethylene glycol and propylene glycol in a quantity to about 30 wt % relative to the total preparation.

The agent may be formulated, for example, as creams, emulsions, gels or also surfactant-containing foaming solutions or other preparations which are suitable for application onto the hair. The preparations preferably exhibit a pH value of 2 to 11. The pH range between 2 and 8 is particularly preferred. Details regarding pH value relate herein to the pH value at 25° C. unless otherwise stated.

The agent may be present in known forms for use. For example, the agent may be formulated in conventional manner as an aerosol hairspray, pump spray, hair gel, hair wax or mousse setting preparation.

If the agent is to be used in the form of an aerosol spray, or aerosol mousse setting preparation, the agent is conventionally filled into a pressure-resistant container and combined with a propellant. The container is finally sealed and provided with a suitable spray device.

Suitable propellants are the compounds conventionally used for this purpose, such as dialkyl ethers, alkanes, N₂O, CO₂ or air. Preferred substances are dimethyl ether and alkanes with 3 to 5 carbon atoms, such as propane, n-butane, iso-butane, n-pentane and iso-pentane; n-butane, propane and mixtures thereof are particularly preferred.

Through the addition of a UV filter, both the agents themselves and the treated fibers may be protected from the harmful effects of UV radiation. Accordingly, in one exemplary embodiment, at least one UV filter is added to the agent. The suitable UV filters are not subject to any general restrictions with regard to structure and physical properties. Rather, any UV filters usable in the field of cosmetics whose absorption maximum is in the UVA (315-400 nm), the UVB (280-315 nm) and/or the UVC (<280 nm) range are suitable. UV filters with an absorption maximum in the UVB range, in particular in the range from approx. 280 to approx. 300 nm, are particularly preferred.

The UV filters suitable for use in the agents may for example be selected from substituted benzophenones, p-aminobenzoic acid esters, diphenylacrylic acid esters, cinnamic acid esters, salicylic acid esters, benzimidazoles and o-aminobenzoic acid esters.

Examples of UV filters suitable for use in the agents are 4-aminobenzoic acid, N,N,N-trimethyl-4-(2-oxoborn-3-ylidenemethyl)aniline methyl sulfate, 3,3,5-trimethylcyclohexyl salicylate (homosalate), 2-hydroxy-4-methoxybenzophenone (benzophenone-3; Uvinul®M 40, Uvasorb®MET, Neo Heliopan®BB, Eusolex®4360), 2-phenylbenzimidazole-5-sulfonic acid and the potassium, sodium and triethanolamine salts thereof, (phenylbenzimidazole sulfonic acid; Parsol®HS; Neo Heliopan® Hydro), 3,3′-(1,4-phenylenedimethylene)-bis(7,7-dimethyl-2-oxobicyclo-[2.2.1]hept-1-ylmethane-sulfonic acid) and the salts thereof, 1-(4-tert.-butylphenyl)-3-(4-methoxy-phenyl)-propane-1,3-dione (butyl methoxydibenzoylmethane; Parsol®1789, Eusolex®9020), α-(2-oxoborn-3-ylidene)-toluene-4-sulfonic acid and the salts thereof, ethoxylated 4-aminobenzoic acid ethyl ester (PEG-25 PABA; Uvinul®P 25), 4-dimethylaminobenzoic acid 2-ethylhexyl ester (octyl dimethyl PABA; Uvasorb®DMO, Escalol®507, Eusolex®6007), salicylic acid 2-ethylhexyl ester (octyl salicylate; Escalol®587, Neo Heliopan®OS, Uvinul®018), 4-methoxycinnamic acid isopentyl ester (isoamyl p-methoxycinnamate; Neo Heliopan®E 1000), 4-methoxycinnamic acid 2-ethylhexyl ester (octyl methoxycinnamate; Parsol®MCX, Escalol®557, Neo Heliopan®AV), 2-hydroxy-4-methoxybenzophenone-5-sulfonic acid and the sodium salt thereof (benzophenone-4; Uvinul®MS 40; Uvasorb®S 5), 3-(4′-methylbenzylidene)-D,L-camphor (4-methylbenzylidene camphor; Parsol®5000, Eusolex®6300), 3-benzylidenecamphor (3-benzylidene camphor), 4-isopropylbenzyl salicylate, 2,4,6-trianilino-(p-carbo-2′-ethylhexyl-1′-oxy)-1,3,5-triazine, 3-imidazol-4-ylacrylic acid and the ethyl esters thereof, polymers of N-{(2 and 4)-[2-oxoborn-3-ylidenemethyl]benzyl}-acrylamide, 2,4-dihydroxybenzophenone (benzophenone-1; Uvasorb®20H, Uvinul®400), 1,1′-diphenylacrylonitrile acid 2-ethylhexyl ester (octocrylene; Eusolex®OCR, Neo Heliopan®Type 303, Uvinul®N 539 SG), o-aminobenzoic acid menthyl ester (menthyl anthranilate; Neo Heliopan®MA), 2,2′,4,4′-tetrahydroxybenzophenone (benzophenone-2; Uvinul®D-50), 2,2′-dihydroxy-4,4′-dimethoxybenzophenone (benzophenone-6), 2,2′-dihydroxy-4,4′-dimethoxybenzophenone-5-sodium sulfonate and 2-cyano-3,3-diphenylacryl acid 2′-ethylhexyl ester. Preference is given to 4-aminobenzoic acid, N,N,N-trimethyl-4-(2-oxoborn-3-ylidenemethyl)aniline methyl sulfate, 3,3,5-trimethyl cyclohexyl salicylate, 2-hydroxy-4-methoxybenzophenone, 2-phenylbenzimidazole-5-sulfonic acid and the potassium, sodium and triethanolamine salts thereof, 3,3′-(1,4-phenylenedimethyleneybis(7,7-dimethyl-2-oxobicyclo-[2.2.1]hept-1-ylmethane-sulfonic acid) and the salts thereof, 1-(4-tert.-butylphenyl)-3-(4-methoxyphenyl)-propane-1,3-dione, α-(2-oxoborn-3-ylidene)-toluene-4-sulfonic acid and the salts thereof, ethoxylated 4-aminobenzoic acid ethyl ester, 4-dimethylaminobenzoic acid 2-ethylhexyl ester, salicylic acid 2-ethylhexyl ester, 4-methoxycinnamic acid isopentyl ester, 4-methoxycinnamic acid 2-ethyl-hexyl ester, 2-hydroxy-4-methoxybenzophenone 5-sulfonic acid and the sodium salt thereof, 3-(4′-methylbenzylidene)-D,L-camphor, 3-benzylidenecamphor, 4-isopropylbenzyl salicylate, 2,4,6-trianilino-(p-carbo-2′-ethylhexyl-1′-oxy)-1,3,5-triazine, 3-imidazol-4-ylacrylic acid and the ethyl esters thereof, and polymers of N-{(2 and 4)-[2-oxoborn-3-ylidenemethyl]benzyl}-acrylamide. Very particularly preferred compounds for use in the agents are 2-hydroxy-4-methoxybenzophenone, 2-phenylbenzimidazole-5-sulfonic acid and the potassium, sodium and triethanolamine salts thereof, 1-(4-tert.-butylphenyl)-3-(4-methoxyphenyl)propane-1,3-dione, 4-methoxycinnamic acid 2-ethylhexyl ester and 3-(4′-methylbenzylidene)-D,L-camphor.

Preferred UV filters are those having a molar extinction coefficient at the absorption maximum of above about 15,000, in particular above about 20,000.

In the case of structurally similar UV filters, it is in many cases the water-insoluble compound which exhibits the greater action in comparison with such water-soluble compounds which differ therefrom by one or more additionally ionic groups. Those UV filters understood herein to be water-insoluble are those which at 20° C. are only about 1 wt. %, in particular no more than about 0.1 wt. %, soluble in water. Furthermore, these compounds should be at least about 0.1, in particular at least about 1 wt. %, soluble in conventional cosmetic oil components at room temperature. The use of water-insoluble UV filters may therefore be preferred.

According to a further embodiment of the invention, preferred UV-filters are those which comprise a cationic group, in particular a quaternary ammonium group.

These UV filters have the general structure U-Q.

The structural element U therein denotes a UV radiation-absorbing group. This group may be derived in principle from the known above-stated UV filters usable in the field of cosmetics, in which a group, generally a hydrogen atom, of the UV filter is replaced by a cationic group Q, in particular with a quaternary amino function.

Compounds from which the structural element U may be derived are for example:
substituted benzophenones;
p-aminobenzoic acid esters;
diphenylacrylic acid esters;
cinnamic acid esters;
salicylic acid esters;
benzimidazoles; and
o-aminobenzoic acid esters.

Structural elements U which are derived from cinnamic acid amide or from N,N-dimethylaminobenzoic acid amide are preferred.

The structural elements U may in principle be selected such that the absorption maximum of the UV filters may lie both in the UVA (315-400 nm) and in the UVB (280-315 nm) or in the UVC (<280 nm) range. UV filters with an absorption maximum in the UVB range, in particular in the range from approximately 280 to approximately 300 nm, are particularly preferred.

Furthermore, the structural element U, also as a function of structural element Q, is preferably selected such that the molar extinction coefficient of the UV filter at the absorption maximum is above about 15,000, in particular above about 20,000.

The structural element Q preferably contains a quaternary ammonium group as the cationic group. This quaternary ammonium group may in principle be linked directly to the structural element U, such that the structural element U is one of the four substituents of the positively charged nitrogen atom. However, one of the four substituents on the positively charged nitrogen atom is preferably a group, in particular an alkylene group with 2 to 6 carbon atoms, which functions as a link between the structural element U and the positively charged nitrogen atom.

Advantageously, the group Q has the general structure —(CH₂)ₓ—N⁺R¹R²R³X⁻, in which x denotes an integer from 1 to 4, R¹ and R² mutually independently denote C₁₋₄ alkyl groups, R³ denotes a C₁₋₂₂ alkyl group or a benzyl group and X⁻ denotes a physiologically acceptable anion. In the context of this general structure, x preferably denotes the number 3, R¹ and R² in each case denote a methyl group and R³ denotes either a methyl group or a saturated or unsaturated, linear or branched, hydrocarbon chain with 8 to 22, in particular 10 to 18, carbon atoms.

Physiologically acceptable anions are for example inorganic anions such as halides, in particular chloride, bromide and fluoride, sulfate ions and phosphate ions and organic anions such as lactate, citrate, acetate, tartrate, methosulfate and tosylate.

Two preferred UV filters with cationic groups are the commercially obtainable compounds cinnamic acid amidopropyltrimethylammonium chloride (Incroquat®UV-283) and dodecyldimethylaminobenzamidopropyldimethyl-ammonium tosylate (Escalol® HP 610).

In one exemplary embodiment, the agents comprise a combination of two or more UV filters. In the context of this embodiment, the combination of at least one water-insoluble UV filter with at least one UV filter having a cationic group is preferred.

The UV filters are conventionally present in quantities of from about 0.01 to about 5 wt. %, relative to the total ready-to-use preparation. Quantities of from about 0.1 to about 2.5 wt. % are preferred.

In one particular embodiment, the agent furthermore contains one or more direct dyes. This makes it possible, when applying the agent, for the treated keratin fibers not only to be structured but also to be dyed at the same time. This may be particularly desirable when only temporary dyeing, for example with conspicuous fashion colors, is desired, which may be removed again from the keratin fiber simply by washing.

Direct dyes are conventionally nitrophenylenediamines, nitroaminophenols, azo dyes, anthraquinones or indophenols. Preferred direct dyes are the compounds known by the international names or trade names HC Yellow 2, HC Yellow 4, HC Yellow 5, HC Yellow 6, HC Yellow 12, Acid Yellow 1, Acid Yellow 10, Acid Yellow 23, Acid Yellow 36, HC Orange 1, Disperse Orange 3, Acid Orange 7, HC Red 1, HC Red 3, HC Red 10, HC Red 11, HC Red 13, Acid Red 33, Acid Red 52, HC Red BN, Pigment Red 57:1, HC Blue 2, HC Blue 11, HC Blue 12, Disperse Blue 3, Acid Blue 7, Acid Green 50, HC Violet 1, Disperse Violet 1, Disperse Violet 4, Acid Violet 43, Disperse Black 9, Acid Black 1, and Acid Black 52 as well as 1,4-diamino-2-nitrobenzene, 2-amino-4-nitrophenol, 1,4-bis-(β-hydroxyethyl)amino-2-nitrobenzene, 3-nitro-4-(β-hydroxyethyl)aminophenol, 2-(2′-hydroxyethyl)amino-4,6-dinitrophenol, 1-(2′-hydroxyethyl)amino-4-methyl-2-nitrobenzene, 1-amino-4-(2′-hydroxyethyl)-amino-5-chloro-2-nitrobenzene, 4-amino-3-nitrophenol, 1-(2′-ureidoethyl)amino-4-nitrobenzene, 4-amino-2-nitrodiphenylamine-2′-carboxylic acid, 6-nitro-1,2,3,4-tetrahydroquinoxaline, 2-hydroxy-1,4-naphthoquinone, picramic acid and the salts thereof, 2-amino-6-chloro-4-nitrophenol, 4-ethylamino-3-nitro-benzoic acid and 2-chloro-6-ethylamino-1-hydroxy-4-nitrobenzene.

Cationic direct dyes are preferably used. Particular preference is here given to:
(a) cationic triphenylmethane dyes, such as for example Basic Blue 7, Basic Blue 26, Basic Violet 2 and Basic Violet 14;
(b) aromatic systems substituted with a quaternary nitrogen group, such as for example Basic Yellow 57, Basic Red 76, Basic Blue 99, Basic Brown 16 and Basic Brown 17; and
(c) direct dyes containing at least one heterocycle which comprises at least one quaternary nitrogen atom, as are for example mentioned in claims 6 to 11 of European patent EP-A2-998 908, to which explicit reference is here made.

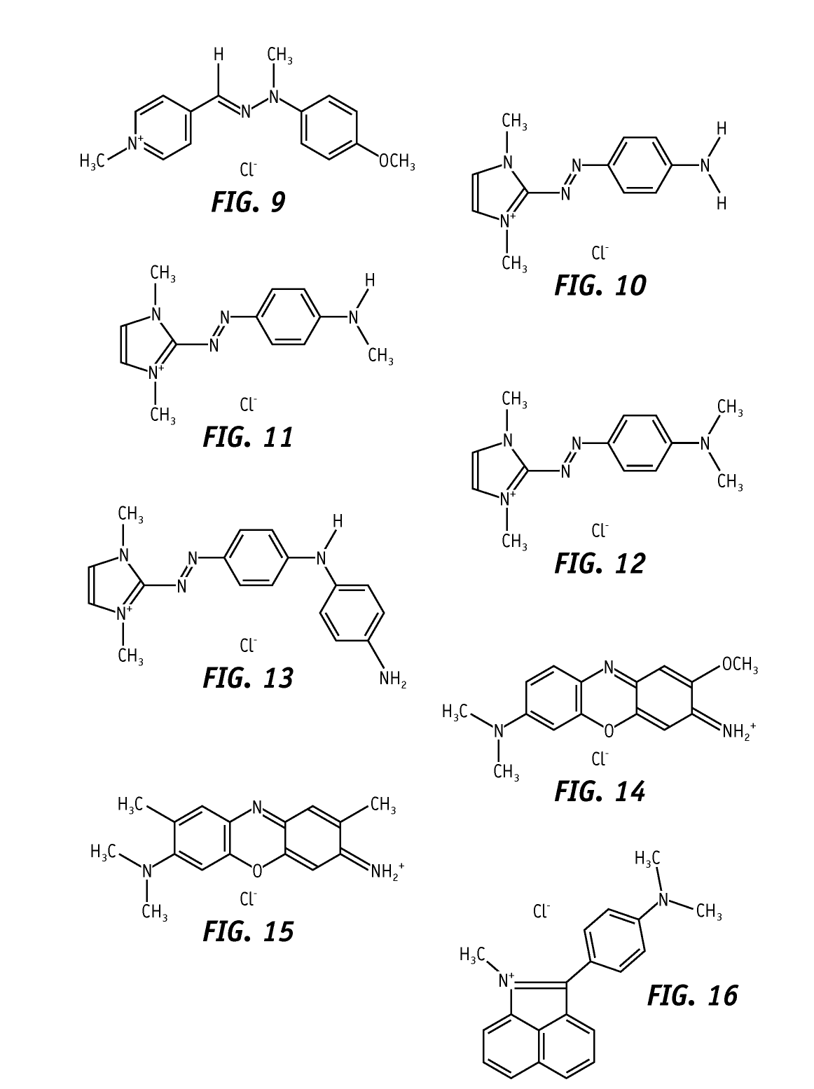

Preferred cationic direct dyes of group (c) are in particular the compounds illustrated in FIGS. 8-16.

The compounds of the formulae of FIGS. 8, 10, and 12, which are also known by the names Basic Yellow 87, Basic Orange 31 and Basic Red 51, are very particularly preferred cationic direct dyes of group (c).

The cationic direct dyes distributed under the trademark Arianor® are cationic direct dyes which are likewise very particularly preferred.

In one exemplary embodiment, the agents contain the direct dyes in a quantity of from about 0.001 to about 20 wt. %, relative to the total agent.

The agents may also contain naturally occurring dyes as are present for example in henna red, henna neutral, henna black, chamomile flowers, sandalwood, black tea, alder buckthorn bark, sage, logwood, madder root, catechu, lotus tree and alkanet root.

It is not necessary for the direct dyes in each case to be uniform compounds. Instead, as a result of the production processes for the individual dyes, the agents contemplated herein may contain subordinate quantities of still further components, provided that these do not have a disadvantageous effect on the styling result or must be excluded for other, for example toxicological, reasons.

In addition to the stated components, the agents may furthermore contain any active ingredients, additives and auxiliary substances known for such preparations.

In many cases, the agents contain at least one surfactant, with not only anionic but also in principle zwitterionic, ampholytic, nonionic and cationic surfactants being suitable. In many cases, however, it has proven advantageous to select the surfactants from among anionic, zwitterionic or nonionic surfactants.

Further active ingredients and auxiliary substances and additives are for example:
thickeners such as agar agar, guar gum, alginates, xanthan gum, gum arabic, gum karaya, locust bean flour, linseed gums, dextrans, cellulose derivatives, for example methylcellulose, hydroxyalkylcellulose and carboxymethylcellulose, starch fractions and derivatives such as amylose, amylopectin and dextrins, clays such as for example bentonite or fully synthetic hydrocolloids, such as for example polyvinyl alcohol;
structuring agents such as maleic acid and lactic acid;
perfume oils, dimethyl isosorbide and cyclodextrins;
quaternized amines such as methyl-1-alkylamidoethyl-2-alkylimidazolinium methosulfate;
defoamers such as silicones;
dyes for coloring the agent;
antidandruff active ingredients such as piroctone olamine, zinc omadine and climbazole;
substances for adjusting pH value, such as for example conventional acids, in particular edible acids and bases;
cholesterol;
consistency providers, such as sugar esters, polyol esters or polyol alkyl ethers;
fats and waxes such as spermaceti, beeswax, montan wax and paraffins;
fatty acid alkanolamides;
complexing agents such as EDTA, NTA, β-alaninediacetic acid and phosphonic acids;
swelling and penetrating substances such as glycerol, propylene glycol monoethyl ether, carbonates, hydrogencarbonates, guanidines, ureas and primary, secondary and tertiary phosphates;
opacifiers such as latex, styrene/PVP and styrene/acrylamide copolymers;
pearlescent agents such as ethylene glycol mono- and distearate as well as PEG-3 distearate;
preservatives; and
antioxidants.

With regard to further optional components and the quantities of these components used, reference is explicitly made to the relevant handbooks known to a person skilled in the art.

Further contemplated herein is the use of a pressure-sensitive adhesive selected from acrylic acid ester copolymers and methacrylic acid ester copolymers in an agent for shaping keratin fibers, in particular human hair, for bringing about or improving remodelability of the shaping achieved or fixed by using the agent.

The pressure-sensitive adhesive is preferably added to the agent for shaping keratin fibers in a quantity such that the total quantity of pressure-sensitive adhesives amounts from about 0.1 to about 10 wt. %, preferably from about 0.5 to about 8 wt. %. The percentages relate to the total agent, less any propellants optionally present in the agent.

Further particular and preferred developments of the use of the agents correspond to the explanations already provided in the description of the agent.

### EXAMPLES

The following Examples are provided for illustration purposes only and are not meant to limit the various embodiments of the present invention in any way.

The quantities are in weight percent unless stated otherwise.

Styling Composition in Gel Form

Agents E1 to E4 may be produced in known manner by mixing the raw materials according to following table.

| Raw materials | E1 | E2 | E3 | E4 |
|---|---|---|---|---|
| Ucar Latex XZ 91964¹ | 2.00 | — | — | — |
| Polytex 6385² | — | 2.00 | — | 1.00 |
| Polytex 6351³ | — | — | 2.00 | 1.00 |
| Synthalen K⁴ | 1.00 | 1.00 | 1.00 | 0.50 |
| PEG-40 Hydrogenated Castor Oil⁵ | 0.40 | 0.40 | 0.40 | 0.40 |
| Perfume | 0.10 | 0.10 | 0.10 | 0.10 |
| Water, deionized | ad 100 | ad 100 | ad 100 | ad 100 |

¹Mixture of butyl acrylate/butadiene copolymer and natural rubber (Dow)
²Acrylic acid ester copolymer (Avery Dennison)
³Acrylic acid ester copolymer (Avery Dennison)
⁴Polyacrylic acid (approx. 89% active substance content; INCI name: Carbomer) (3V Group, Italy)
⁵Hydrogenated castor oil with approx. 40-45 EO units (INCI name: PEG-40 Hydrogenated Castor Oil) (BASF, Germany)

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

1. An agent for shaping keratin fibers containing in a cosmetically acceptable carrier at least one pressure-sensitive adhesive selected from the group consisting of acrylic acid ester copolymers and methacrylic acid ester copolymers.

2. The agent as claimed in claim 1, wherein the pressure-sensitive adhesive is selected from acrylic acid butyl ester-butadiene copolymers.

3. The agent of claim 1, wherein pressure-sensitive adhesives are used which remain in the fibers even after rinsing the fibers with water at a temperature of 30° C. for in each case 2 minutes, the water being used in each rinsing operation in a quantity such that the weight ratio of fibers to water amounts to 1:10.

4. The agent of claim 1, wherein the pressure-sensitive adhesive is present in a quantity of from about 0.1 to about 10 wt. %.

5. The agent of claim 1, further comprising a film-forming and setting polymer, other than the pressure-sensitive adhesive, in a quantity of from about 0.1 to about 20 wt. %.

6. The agent of claim 5, wherein the film-forming and setting polymer is selected from the group consisting of:
aminomethylpropanol salts of copolymers of allyl methacrylate with one or more monomers selected from acrylic acid, methacrylic acid, acrylic acid esters and methacrylic acid esters;
vinylpyrrolidone/vinyl acetate copolymers;
vinylpyrrolidone/vinylcaprolactam/dimethylaminopropylacrylamide copolymers;
copolymers of octylacrylamide with t-butylaminoethyl methacrylate and at least two monomers selected from acrylic acid, methacrylic acid, acrylic acid esters and methacrylic acid esters; and
copolymers of C₁₋₂ alkyl succinates with hydroxyalkyl acrylates and at least one monomer selected from acrylic acid, methacrylic acid, acrylic acid esters and methacrylic acid esters.

7. The agent of claim 1, wherein the cosmetically acceptable carrier comprises an aqueous, an alcoholic or an aqueous-alcoholic medium.

8. A method for shaping keratin fibers, the method comprising the steps of applying to the keratin fibers an agent comprising a pressure sensitive adhesive selected from acrylic acid ester copolymers and methacrylic acid ester copolymers.

9. The agent of claim 4, wherein the pressure-sensitive adhesive is present in a quantity of from about 0.5 to about 8 wt. %.

10. The agent of claim 5, wherein the film-forming and setting polymer is present in a quantity of from about 0.5 to about 15 wt. %.

11. The agent of claim 10, wherein the film-forming and setting polymer is present in a quantity of from about 1.0 to about 10 wt. %.
